(19) United States
(12) Patent Application Publication
(10) Pub. No.: US 2026/0011409 A1
(43) Pub. Date: Jan. 8, 2026

(54) SYSTEM AND METHOD FOR RRAM-BASED GENOME SEQUENCING

(71) Applicants: Deliang Fan, Baltimore, MD (US); Fan Zhang, Baltimore, MD (US)

(72) Inventors: Deliang Fan, Baltimore, MD (US); Fan Zhang, Baltimore, MD (US)

(73) Assignee: Arizona Board of Regents on behalf of Arizona State University, Scottsdale, AZ (US)

(21) Appl. No.: 19/257,604

(22) Filed: Jul. 2, 2025

Related U.S. Application Data
(60) Provisional application No. 63/667,343, filed on Jul. 3, 2024.

(51) Int. Cl. G16B 30/10; G16B 50/30

(52) U.S. Cl. CPC G16B 30/10; G16B 50/30

(57) ABSTRACT

A system for calculating DNA short-read alignment comprises a memory comprising a plurality of memory cells, the memory comprising first and second regions, a plurality of transmission gates, a plurality of sense amplifiers, each having first and second outputs connected to each bitline in the memory, a plurality of logic gates, each connected to the first and second outputs of each sense amplifier, a digital peripheral circuit connected to the outputs of the logic gates, and a processor configured to perform steps comprising performing a Burrows-Wheeler transform on a nucleotide sequence represented as a string, storing the transformed nucleotide sequence in the first region of the memory, storing a short-read nucleotide sequence in the second region of the memory, activating first and second rows of the memory, and calculating DNA short-read alignment using the digital peripheral circuit.

### CROSS-REFERENCE TO RELATED APPLICATIONS

This application claims priority to U.S. Provisional Patent Application No. 63/667,343, filed on Jul. 3, 2024, incorporated herein by reference in its entirety.

### STATEMENT REGARDING FEDERALLY SPONSORED RESEARCH OR DEVELOPMENT

This invention was made with government support under 1652866, 2003749, and 2144751 awarded by the National Science Foundation. The government has certain rights in the invention

### BACKGROUND OF THE INVENTION

Next-generation sequencing (NGS) technologies enable rapid and accurate determination of nucleotide (nt) sequences within genomes, empowering disease diagnostics, cancer risk assessment, tailored patient treatments, prenatal testing, and a wide range of other personalized medicine approaches. NGS platforms can generate terabytes of DNA sequence data (e.g., short reads) in a single run. These short reads do not come with position information relevant to the overall genome and must be aligned to a reference genome before further genomic analysis or scientific discovery. However, the human reference genome is huge, containing approximately 3.2 billion nucleotide bases (A, C, G, T) (see H. Li et al. Bioinformatics, 2009) Thus, a major challenge in sequencing is to map the short reads from NGS to the overall human reference genome.

State-of-the-art (SOTA) alignment processes still require hours or days to align large volumes of short read data, even using very powerful CPUs/GPUs (see M. Alser et al. IEEE Micro, 2020). This is mainly due to the off-chip bandwidth limitations and inefficiencies of moving big data between computation and memory units, i.e., the memory-wall challenge. It is widely known that the bottleneck for the entire genomic analysis process is alignment of DNA short reads, which is memory- and compute-intensive (see S. Angizi et al. DAC, 2019; and F. Zhang et al. IEEE JETCAS, 2023). To address the memory-wall challenge, Computing-in-Memory (CIM) has gained significant interest owing to its high energy efficiency and superior throughput (see B. Li et al. IEEE TCAD, 2015), and has been widely investigated for accelerating AI applications (see I. Yeo et al. Nature Electronics, 2022; and A. Sridharan et al. ESSCIRC, 2022) but has not been applied considerably for genome alignment.

Disclosed herein is a resistive random access memory (RRAM) based CIM macro chip prototype for SOTA Burrows-Wheeler Transformation (BWT) (see H. Li et al. Bioinformatics, 2009; M. Alser et al. IEEE Micro, 2020; M. Burrows et al. Digital Equipment Corporation technical reports, 1994; and Y.-C. Wu et al. IEEE TBioCAS, 2017) based genome sequencing alignment applications. The designed CIM macro supports all core instructions, i.e., XNOR based match, count, and addition, required by alignment algorithms. As designed, this approach could work independently as a parallel ‘alignment core’ that could process local correlated reference genomic data to significantly improve system parallelism and throughput. Leveraging the multi-bit property of RRAM cells, the disclosed in-memory XNOR-based match circuits are flexible to support both 1- and 2-bit per cell encoding of nucleotides (A, C, T, G). The CIM macro was implemented in a prototype chip that monolithically integrates HfO₂ RRAM and 65 nm CMOS, achieving the best energy efficiency to date with 2.07 TOPS/W and 2.12 G suffixes/J at 1.0V.

### SUMMARY OF THE INVENTION

In one aspect, a system for calculating DNA short-read alignment comprises a memory comprising a plurality of memory cells arranged in columns and rows, each column having a bitline, the memory comprising first and second regions, a plurality of transmission gates, each being connected to a row in the memory, a plurality of sense amplifiers, each having first and second outputs connected to each bitline in the memory, a plurality of logic gates, each connected to the first and second outputs of each sense amplifier, a digital peripheral circuit connected to the outputs of the logic gates, and a processor communicatively connected to the memory, configured to perform steps comprising performing a Burrows-Wheeler transform on a nucleotide sequence represented as a string, storing the transformed nucleotide sequence in the first region of the memory, storing a short-read nucleotide sequence in the second region of the memory, activating first and second rows of the memory via first and second transmission gates of the plurality of transmission gates, wherein the first row is in the first region of the memory and the second row is in the second region of the memory, and calculating DNA short-read alignment using the digital peripheral circuit.

In one embodiment, the memory is a resistive random-access memory (RRAM). In one embodiment, the digital peripheral circuit comprises a plurality of latches, each connected to an output of a logic gate. In one embodiment, the digital peripheral circuit comprises an adder connected to the latches. In one embodiment, the logic gates are AND gates. In one embodiment, each memory cell comprises a memristor, and the memristors form a voltage divider with the corresponding bitline when the transmission gates activate the first and second rows.

In one embodiment, the memory, the transmission gate, the sense amplifiers, the logic gates, and the digital peripheral circuit are all positioned on the same integrated circuit. In one embodiment, the processor is positioned on the same integrated circuit. In one embodiment, the first transmission gate is configured to connect the first memory row to a source voltage line, and the second transmission gate is configured to connect the second memory row to a ground. In one embodiment, the memory further comprises a third region, and wherein the processor is configured to store a marker table having a counter for each nucleotide.

In one aspect, a method of calculating DNA short-read alignment comprises providing a memory and a processor, performing a Burrows-Wheeler transform on a nucleotide sequence represented as a string, storing the transformed nucleotide sequence in the memory, storing a short-read nucleotide sequence in the memory, activating first and second rows of the memory wherein the first row holds a portion of the transformed nucleotide sequence and the second row holds a portion of the short-read nucleotide sequence, and calculating DNA short-read alignment using voltage levels measured at the bitlines in the memory.

In one embodiment, the memory is a resistive random-access memory (RRAM). In one embodiment, the method further comprises the step of calculating whether first and second nucleotides stored in the first and second rows are the same by calculating a logical XNOR of the voltage values measured at the bitlines. In one embodiment, the method further comprises the step of storing the result of each logical XNOR in a latch. In one embodiment, the method further comprises adding the values stored in the latches using an adder. In one embodiment, each memory cell in the memory comprises a memristor, and the method comprises the step of creating a voltage divider with the memristors in the first and second row of each column in the memory.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 is a diagram of pre-computed tables for BWT-based alignment.

FIG. 2A and FIG. 2B is a diagram of dataflow of alignment in one compute-in-memory macro.

FIG. 3A, FIG. 3B, and FIG. 3C are diagrams of the compute-in-memory macro architecture and circuits.

FIG. 4A and FIG. 4B are graphs of RRAM resistance distribution and sensing voltage.

FIG. 5A is a graph of maximum frequency and throughput versus a range of supply voltages.

FIG. 5B is a graph of energy efficiency and digital energy efficiency versus a range of supply voltages.

FIG. 6 is a graph of macro area breakdown.

FIG. 7 is a chip micrograph and layout of the disclosed device with modules labelled.

FIG. 8 is an image of an exemplary experimental setup.

FIG. 9 is a diagram of an exemplary computing device.

### DETAILED DESCRIPTION

It is to be understood that the figures and descriptions of the present invention have been simplified to illustrate elements that are relevant for a clear understanding of the present invention, while eliminating, for the purpose of clarity, many other elements found in related systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C #, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

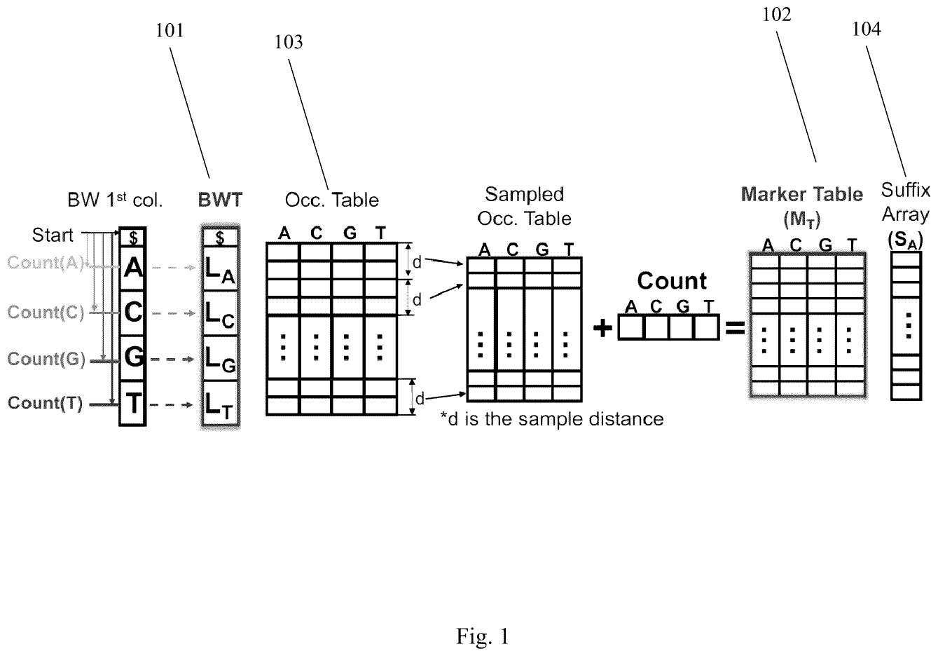

Prior work (S. Angizi et al. DAC, 2019; and F. Zhang et al. IEEE JETCAS, 2023), has disclosed a CIM-friendly DNA short read alignment algorithm, called alignment-in-memory as shown in Algorithm 1 below, which recursively uses digital bit-wise logic functions to implement the fundamental computing core of BWT and FM-Index based genome alignment algorithms (see H. Li et al. Bioinformatics, 2009; M. Burrows et al. Digital Equipment Corporation technical reports, 1994). Additional information about CIM-friendly DNA short read systems may be found in U.S. patent application Ser. No. 18/187,203, filed Mar. 21, 2023, incorporated herein by reference. Similar to the original algorithm, a one-time pre-computation is needed based on the reference genome S to construct required reference tables as shown in FIG. 1. The BWT is a reversible rearrangement of a character string. Exact alignment finds all occurrences of the short read R (m bp) in the reference genome S (n bp). Note that only the BWT 101 and Marker Table (M_{T}) 102 are the primary genome alignment computations needed, and thus need to be stored in the disclosed CIM macro. Other tables, like the occurrence table (Occ. table) 103 and Suffix Array (S_{A}) 104, are only related to pre- or post-processing of the core alignment function. The BWT 101 and M_{T} 102 table mapping are one-time write and only memory-read based operations are needed during alignment computation, and are readily implemented with non-volatile RRAM technology.

Algorithm 1

Require: Pre-Compute and Data Mapping: Partition pre-computed BWT, Marker Table
(M_{T} ) and Suffix Array (S_{A}).

| | input: Genome Short Read-R | |
|---|---|---|
| | output: Positions of short read-R in reference genome-S | |
| | Step-1. Initialization: | |
| 1: | low ← 0, high → S − 1 | |
| | Step-2. Backward Search: | |
| 2: | for i := \|R\| − 1 to 0 do | |
| 3: | low ← Bound(M_{T} [ low/d ], R[i], low) | |
| 4: | high ← Bound(M_{T} [ high/d ], R[i], high) | |
| 5: | if low ≥ high then | |
| 6: | break & return 0 | [symbol] There is no exact alignment |
| 7: | end if | |
| 8: | end for | |
| | Step-3. Get matched positions from stored suffix array based on a search result: | |
| 9: | for j := low to high − 1 do | |
| 10: | positions ← MEM(S_{A}[j]) | [symbol] Read positions from Suffix Array memory |
| 11: | end for | |
| | Define procedure Bound: | |
| 12: | Procedure: Bound(MT, nt, id) | [symbol] compute matched interval |
| 13: | count_match ← 0 | |
| 14: | for j := 0 to j < (id mod d) do | [symbol] count number of nt within the BWT region |
| 15: | if XNOR_Match (nt, BWT [id − (id mod d) + j]) == 1 then | |
| 16: | count_match = count_match + 1 | |
| 17: | end if | |
| 18: | end for | |
| 19: | marker ← MEM(MT [ id/d ], nt) | [symbol] Read Marker Table value |

| | |
|---|---|
| 20: | return ADD(marker, count_match) |
| 21: | end Procedure |

The process described in Algorithm 1 is mainly implemented through the main Bound (M_{T}, nt, id) procedure performed on BWT, which computes the updated interval bound (either low or high) value from M_{T} with bucket width d, input index-id and input nucleotide-nt. Such a procedure is iteratively used in every step of the ‘for’ loop. To make the algorithm hardware-friendly for the CIM platform, computations mainly leverage logic functions, e.g., XNOR_Match and ADD. XNOR_Match conducts a parallel in-memory match operation to determine if the current input-nt matches with BWT elements stored in current memory, and then updates the count_match (i.e., counter) based on matching result (lines 14 to 18 in Algorithm 1). ADD performs a 32-bit integer (determined by the 3.2 billion reference genome length) addition operation to implement ‘marker+count_match’, then the computed sum is returned as the main Bound function output (line 20). In summary, to implement all the alignment-related computations in Algorithm 1, one embodiment of the CIM platform supports parallel XNOR operations between input-nt and decoded BWT elements (line 15), counts the XNOR results (line 16), reads the marker from marker table (line 19), and adds it to the current counter value (line 20). The updated count is in some embodiments considered the final result of the macro, and is then sent out of the macro for post-processing, for example on an integrated processor core or external micro-controller.

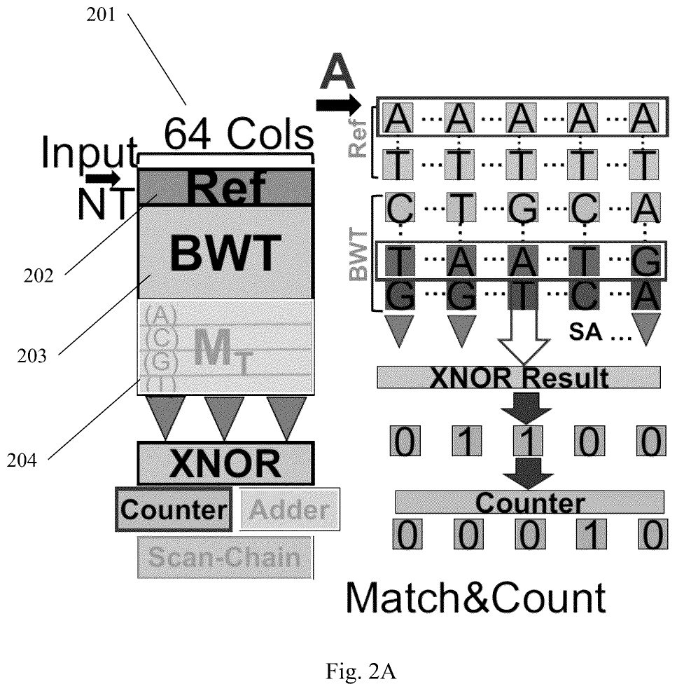

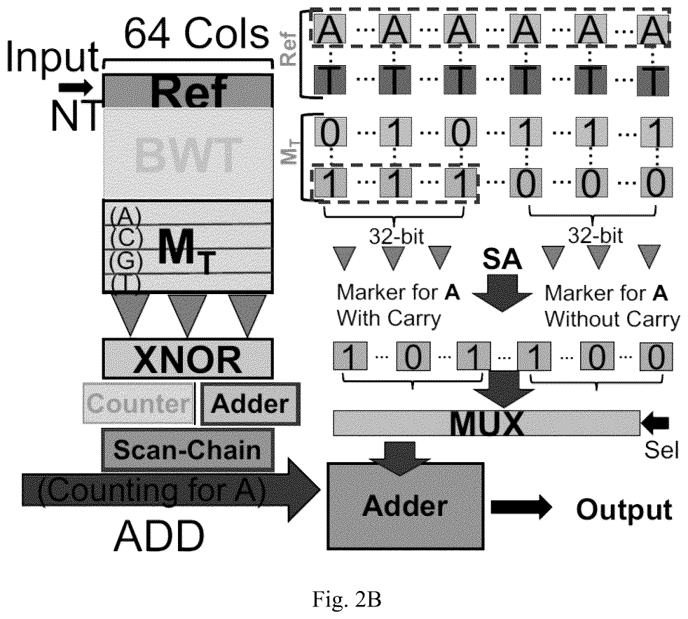

Preliminary work has developed a correlated data partition and memory mapping methodology that could partition the BWT and MT tables based on the target CIM macro memory size to guarantee each macro could work independently as an alignment-core to process within a local memory array with correlated data partitions. More details about the data partition algorithm may be found in S. Angizi et al. DAC, 2019 and F. Zhang et al. IEEE JETCAS, 2023. FIG. 2A and FIG. 2B show the data mapping and dataflow of alignment. For each CIM macro, the memory array 201 is divided into three zones for storing and processing three data types: i) rows [0:3] defined as Ref (202) which comprises four rows of memory, one row of the four being programmed entirely with each of a single nucleotide selected from [A,C,G,T] as a compute reference; ii) rows [4:15] storing the BWT partition (203) for the current CIM macro; iii) rows [16:63] (204) storing the MT table partition.

In some embodiments, row 0 of the Ref region 202 is the A row (Adenine), row 1 of the Ref region is the T row (Thymine), row 2 of the Ref region is the Crow (Cytosine) , and row 3 of the Ref region is the G row (Guanine). In other embodiments, the nucleotides in the Ref region may be stored in a different order. In some embodiments, the reference zone may be longer than four rows, and additional nucleotides other than A/T/C/G may be stored in other rows, including but not limited to U, R, Y, K, M, S, W, B, D, H. V, N, or any other nucleotide.

As illustrated in FIG. 2A and FIG. 2B, the core alignment process in one macro requires two main stages: match &count and ADD.

The match&count stage includes the parallel in-memory XNOR_Match and counting the matching result using a digital counter. For XNOR_Match operation, the first operand is the input-nt (e.g. A/T/C/G), where the corresponding row in Ref region will be activated representing current Bound function input. The second operand is a sub-list of BWT elements decoded by index-id and d (line-15 in algorithm). Therefore, in this stage, two decoded rows (one from Ref and one from the BWT region) are activated to implement a parallel XNOR based match and count outputs (lines 14 to 18). In the following ADD stage, the corresponding marker value from the MT (line-19) is fetched and added to the current counter (line 20) through a digital adder. Since the RRAM array only has 64 columns in the macro, the counting result will not be greater than 64 which can be represented in 6 bits. Performing a 32-bit addition with a 6-bit number in each local CIM macro is unnecessary. In one embodiment of the disclosed design one 6-bit adder is used and the bias based on the index of the current CIM macro is calculated using a similar BWT and MT partition algorithm (see S. Angizi et al. DAC, 2019). Note that this pre-calculation is also performed one time and saved within the MT region for each type of nucleotide. Finally, the ADD result is returned as the main Bound function output, which is used during the processing of the next nucleotide in the same short read.

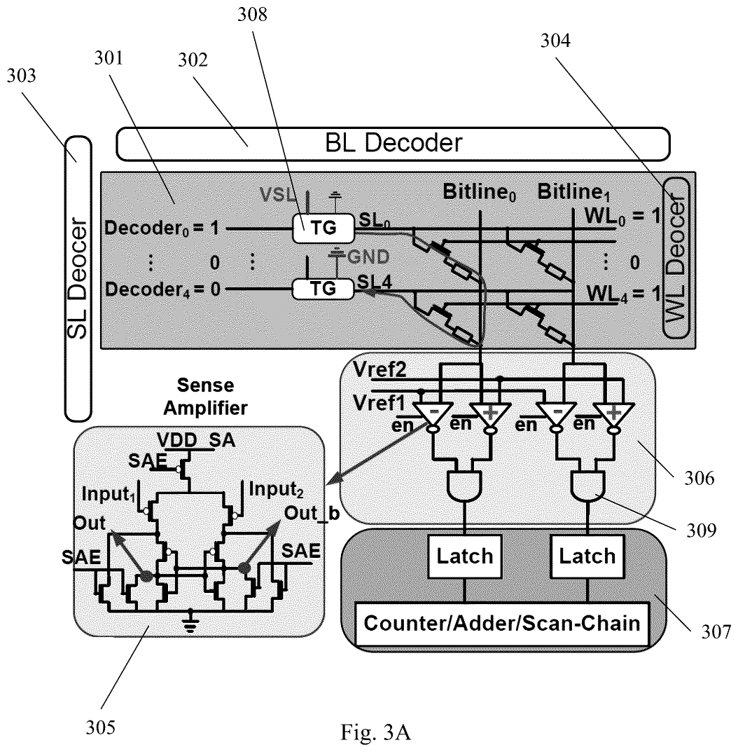

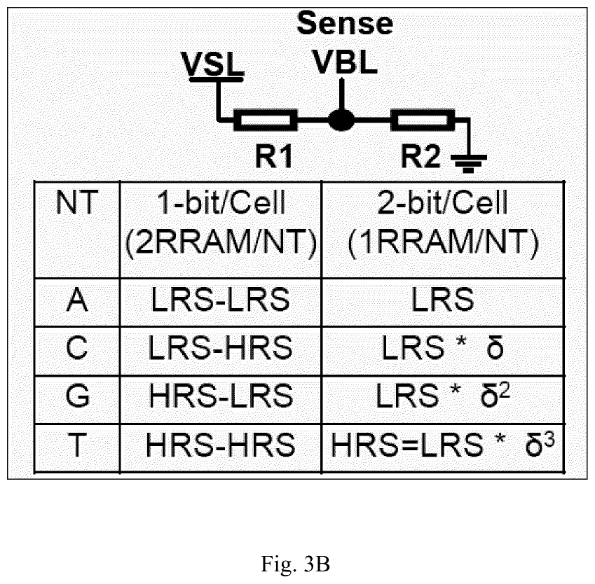

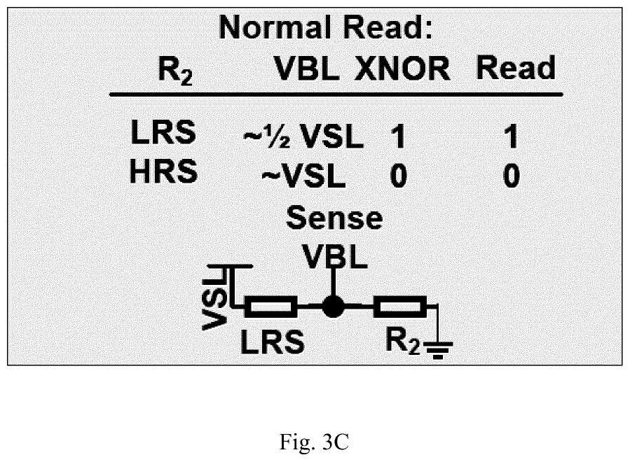

FIG. 3A-FIG. 3C show the proposed architecture and circuits of one CIM macro to perform alignment operations. The computational array comprises one 64×64 RRAM array 301, Source Line (SL) decoder 303, Word Line (WL) decoder 304, Bitline (BL) decoder 302, sense amplifier (SA) 305, transmission gates (TG) 308, level shifter, etc. The depicted array further comprises a digital peripheral circuit 307 which comprises latches to temporarily buffer the output of the logic gates and a counter/adder/scan-chain to post-process the calculated values. As described earlier, for the XNOR_match operation, two rows are simultaneously activated, for example as shown in FIG. 3A. This forms a voltage divider circuit in each BL, where the BL voltage is determined by the two activated RRAM cells in the same column. Two complementary TGs 308 controlled by the SL decoder 303 and drivers are used to provide the operating voltages. The first TG 308 corresponding to the first XNOR operand, given an input-nt in the Ref region, connects to the source voltage line (VSL). While the other TG corresponding to the second XNOR operand, BWT, connects to the GND, for forming the voltage divider circuit as shown in FIG. 3B.

With reference to FIG. 3B, the voltage at the BL (VBL) should be around the middle of the supplied voltage (VSL) when the resistance of R1 is equal or very close to R2, meaning a ‘match’ is found. Otherwise, it is ‘not matched’. To achieve such a matching function, two sense amplifiers (SA) are used as voltage comparators per BL, where they share the same BL but with different reference voltages: Vref1 and Vref2. An AND logic gate 309 is connected to the output of two Sas 305, so that it only outputs ‘1’ when Vref1<VBL<Vref2, thus implementing an XNOR-based matching function. In some embodiments, Vref1 and Vref2 may be selected as bounds around a midpoint of the VSL. In one embodiment, where the VSL is 400 mV, Vref1 may be about 100 mV, about 110 mV, about 120 mV, about 130 mV, about 140 mV, about 150 mV, about 160 mV, about 170 mV, about 180 mV, or about 190 mV. Vref2 may be about 210 mV, about 220 mV, about 230 mV, about 240 mV, about 250 mV, about 260 mV, about 270 mV, about 280 mV, about 290 mV, or about 300 mV. In some embodiments, Vref1 and Vref2 may be selected to be about the same distance from the midpoint of VSL, for example in one embodiment Vref1 may be about 130 mV and Vref2 may be about 270 mV. In some embodiments, one of Vref1 or Vref2 may be further from the midpoint, for example Vref1 may be about 130 mV and Vref2 may be about 280 mV. In other embodiments, where the VSL is higher or lower, the Vref1 and Vref2 may correspondingly be higher or lower in a manner proportional to VSL in order to maintain boundaries around the midpoint of VSL, for example ±5%, ±7%, ±10%, ±12%, ±15%, ±17%, ±20%, or the like.

As described earlier, the example R1 and R2 here represent the two nucleotides being compared, as such, each column outputs ‘1’ when the two nucleotides being compared are the same. With the operation being independent of other columns, it enables 64 parallel matching operations in one cycle. The proposed design supports both a 1-bit/cell and/or 2-bit/cell XNOR_match, where the sense margin is mainly dependent on the RRAM's on/off ratio, variation, resistance difference between different encoded levels and VSL. For the 1-bit/cell case, each nucleotide requires two adjacent RRAM cells for encoding, whereas in the 2-bit/cell case, each RRAM cell can be programmed into four different resistance levels: low resistor state (LRS), LRSδ, LRSδ², and high resistor state (HRS)=LRSδ³ to represent the 4 different nucleotides.

In the disclosed design, to support independent RRAM programming, two complementary TGs are also present on BLs. During the XNOR_match operation, the BL is connected to SAs through the TGs. While, during RRAM cell programming, the BL is disconnected from SAs. The column decoder assigns the selected BL to an analog IO pad that provides Form/Set/Reset pulses to arbitrary RRAM cells in the array. Note that the VWL/VSL/VBL are directly connected to different analog IO pads to provide arbitrary pulses for RRAM device programming and testing.

The read of the marker value stored in the MT memory region (line 19 in the algorithm) leverages the existing two row activation scheme and XNOR_match circuit, but one of them must be in an all-LRS state. As illustrated in FIG. 3C, it can be seen that when R1 is in the LRS state, the XNOR_match output is the equivalent of reading R2's status. In both 1-bit and 2-bit per-cell cases, the first row is always programmed at the all-LRS state to encode the nucleotide ‘A’, as shown in FIG. 3B. Thus, a read operation is accomplished by activating the first row (or whichever of the reference rows is encoded at an all-LRS state) and the row which needs to be read.

### Exemplary Computing Device

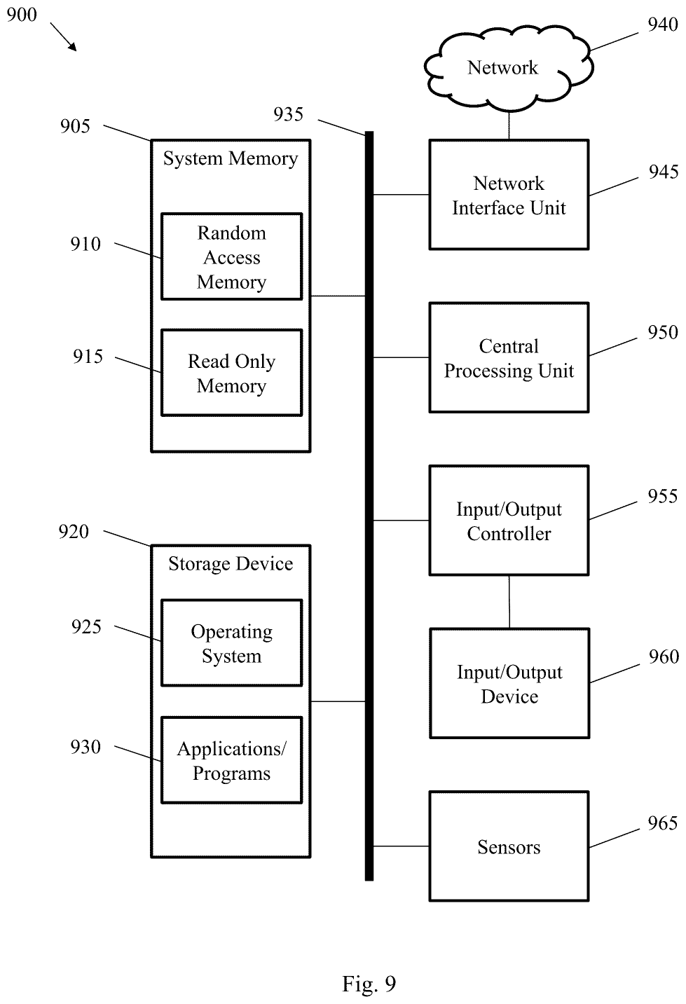

FIG. 9 and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 9 depicts an illustrative computer architecture for a computer 900 for practicing the various embodiments of the invention. The computer architecture shown in FIG. 9 illustrates a conventional personal computer, including a central processing unit 950 (“CPU”), a system memory 905, including a random access memory 910 (“RAM”) and a read-only memory (“ROM”) 915, and a system bus 935 that couples the system memory 905 to the CPU 950. A basic input/output system containing the basic routines that help to transfer information between elements within the computer, such as during startup, is stored in the ROM 915. The computer 900 further includes a storage device 920 for storing an operating system 925, application/program 930, and data.

The storage device 920 is connected to the CPU 950 through a storage controller (not shown) connected to the bus 935. The storage device 920 and its associated computer-readable media provide non-volatile storage for the computer 900. Although the description of computer-readable media contained herein refers to a storage device, such as a hard disk or CD-ROM drive, it should be appreciated by those skilled in the art that computer-readable media can be any available media that can be accessed by the computer 900.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 900 may operate in a networked environment using logical connections to remote computers through a network 940, such as TCP/IP network such as the Internet or an intranet. The computer 900 may connect to the network 940 through a network interface unit 945 connected to the bus 935. It should be appreciated that the network interface unit 945 may also be utilized to connect to other types of networks and remote computer systems.

The computer 900 may also include an input/output controller 955 for receiving and processing input from a number of input/output devices 960, including a keyboard, a mouse, a touchscreen, a camera, a microphone, a controller, a joystick, or other type of input device. Similarly, the input/output controller 955 may provide output to a display screen, a printer, a speaker, or other type of output device. The computer 900 can connect to the input/output device 960 via a wired connection including, but not limited to, fiber optic, Ethernet, or copper wire or wireless means including, but not limited to, Wi-Fi, Bluetooth, Near-Field Communication (NFC), infrared, or other suitable wired or wireless connections.

As mentioned briefly above, a number of program modules and data files may be stored in the storage device 920 and/or RAM 910 of the computer 900, including an operating system 925 suitable for controlling the operation of a networked computer. The storage device 920 and RAM 910 may also store one or more applications/programs 930. In particular, the storage device 920 and RAM 910 may store an application/program 930 for providing a variety of functionalities to a user. For instance, the application/program 930 may comprise many types of programs such as a word processing application, a spreadsheet application, a desktop publishing application, a database application, a gaming application, internet browsing application, electronic mail application, messaging application, and the like. According to an embodiment of the present invention, the application/program 930 comprises a multiple functionality software application for providing word processing functionality, slide presentation functionality, spreadsheet functionality, database functionality and the like.

The computer 900 in some embodiments can include a variety of sensors 965 for monitoring the environment surrounding and the environment internal to the computer 900. These sensors 965 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the system and method of the present invention. The following working examples therefore, specifically point out the exemplary embodiments of the present invention, and are not to be construed as limiting in any way the remainder of the disclosure.

### Results

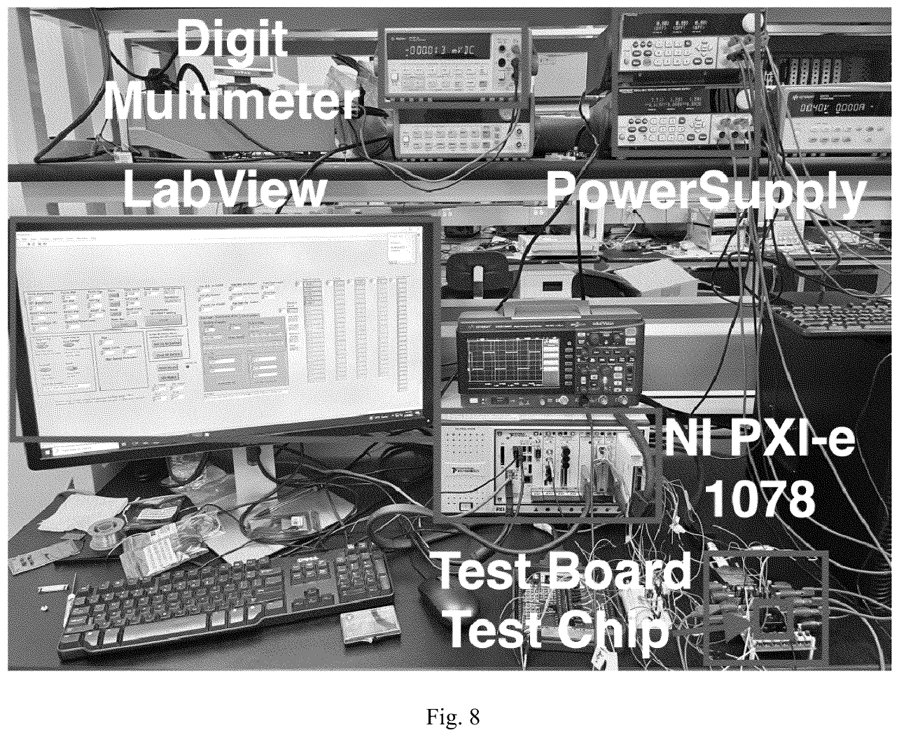

The disclosed chip was fabricated using a custom 65 nm CMOS process with integration of H fO₂ RRAM between M1 and M2 using a 300 mm wafer platform. More detailed device-level RRAM characteristics and fabrication process were reported in a prior publication (J. Hazra et al. IMW, 2021). As shown in FIG. 8, the automated process of the FORM/SET/RESET/READ operations were developed for the RRAM array. Repeatable pulses were sent from SL/BL to BL/SL for each device during the programming process until the targeted resistance level was achieved, or the writing attempts reached the maximum limit. The WL was used for address indexing of the 1T1R cell in the RRAM array, and the typical value for programming amplitude (V), pulse width (PW), and gate control voltage (V WL) are listed below:

FORM: V_{form}=3.8V, PW=10 μs, V WL=1.8V, repeat limit=50 times; 2) SET: Vₛₑₜ=1.2V, PW=1us, V WL=1.5V, target resistance value was <3 kΩ; 3) RESET: Vᵣₑₛₑₜ=3.3V, PW=100 ns, V WL=3.3V, target resistance value was >50 kΩ; READ: V_{read}=0.2V, V WL=3.3V.

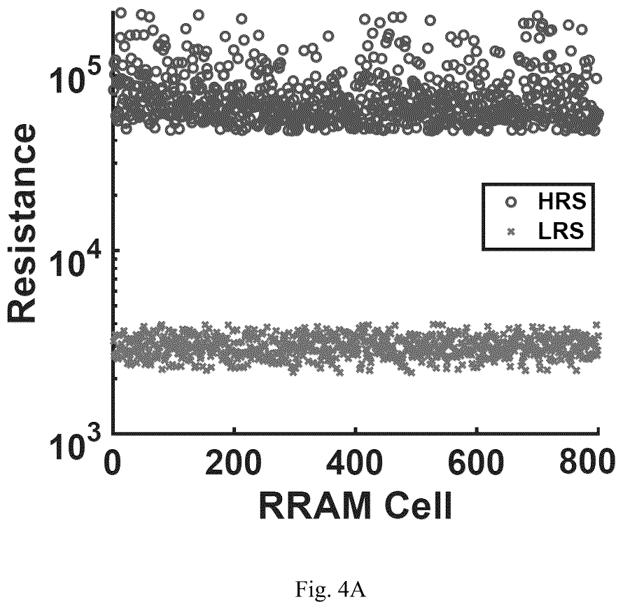

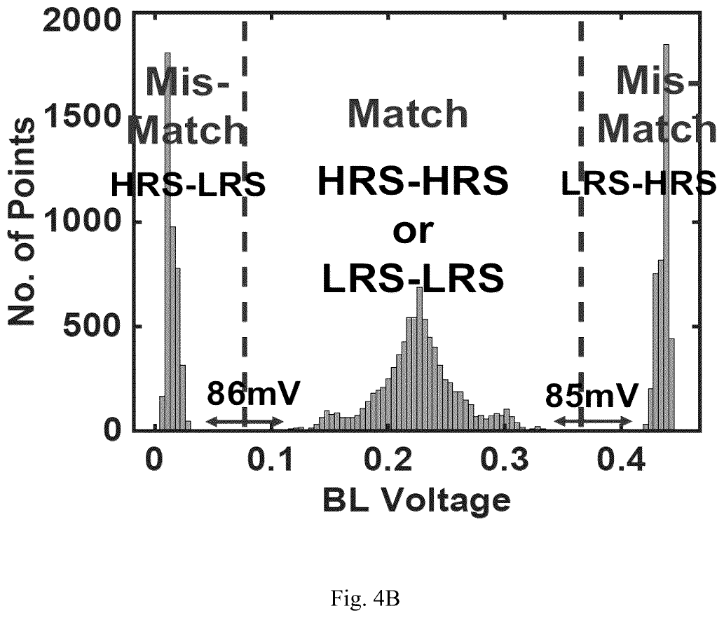

In the disclosed experimental example, the measurement results of a 1-bit/cell RRAM scheme were tested. FIG. 4A shows the measured RRAM LRS/HRS distribution across 5 test chips and the corresponding pattern match voltage distribution is shown in FIG. 4B, where the center voltage distributions represent the BL voltage values with ‘MATCH’ results. For the XNOR_match operation, the VSL voltage is up-bounded to 0.45V. As observed, a voltage higher than 0.45V may disturb RRAM resistance during inference operation.

The chip's core power consumption comes from two main sources: analog input and digital power supply. The analog input feeds in from the SL through the given path of RRAM devices, with a fixed power supply at 0.45 V, to maximize the sensing margin while still preventing RRAM cells from performing destructive read operations. Analog power varies with test vectors from 150 μW to 400 μW, as a result of different numbers of HRS and LRS in the circuit paths. In the energy efficiency calculation, 250 μW was used as the average analog power, where at this point the HRS and LRS cells are 50% each in the test vectors.

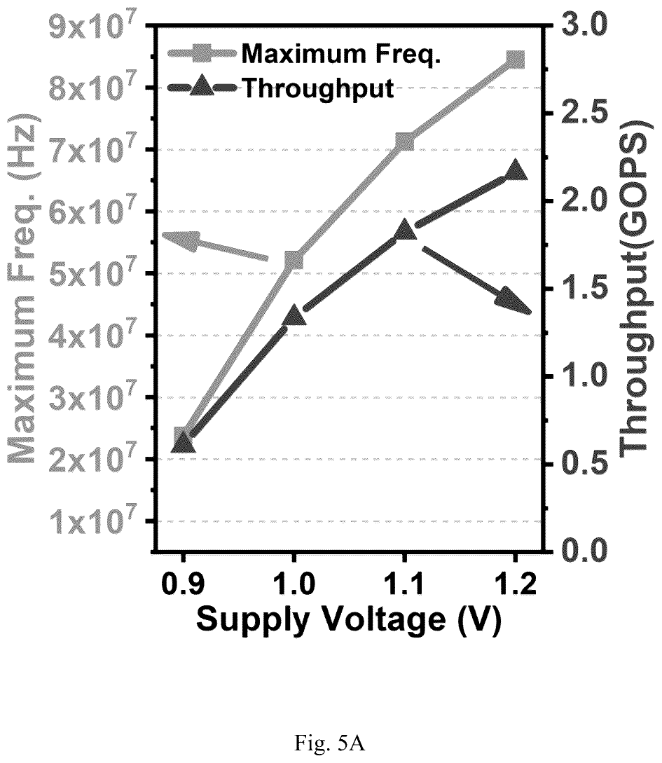

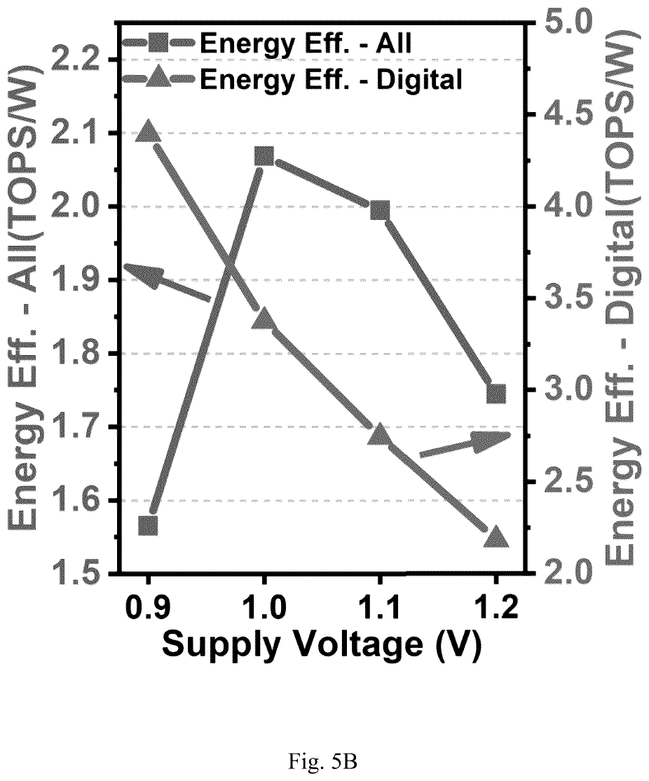

The digital power includes the digital decoder, clock generator, digital driver for WL/SL/BL, and SAs. The digital power strongly correlates with the supply voltage and operating frequency. A voltage sweep was performed for the digital circuits from 0.9 V to 1.2 V, to explore the optimal voltage for the highest energy efficiency and the maximum frequency, and the results are shown in FIG. 5A and FIG. 5B. In FIG. 5A, the maximum frequency and throughput with voltage scaling are shown. The maximum frequency (f_{MAX}) indicates the highest frequency at each supply voltage where all the circuit functions remain correct.

The definition of throughput in this work is: OPs/t×f_{MAX}, where OPs is the number of operations in one XNOR_match operation. For the purposes of this experiment, OPs is 128 total operations, comprising 64 XNOR operations and 64 counting operations (summing 64 1-bit numbers). t is the required number of cycles for the circuits to process the outputs from the RRAM array, which is 5 in this work for the SAs and the parallel adder.

As shown in FIG. 5A, at 1.2V supply, a maximum frequency of 84.5 MHz and maximum throughput of 2.16 GOPS (billions of operations per second) were achieved. As the supply voltage decreased, the frequency and throughput decreased largely linearly.

FIG. 5B shows the digital energy efficiency and overall (including digital and analog parts) energy efficiency. As the supply voltage decreases, the digital energy efficiency increases, while the analog energy efficiency degrades due to lower maximum frequency. The overall energy efficiency, which combines both digital and analog parts, reaches its maximum value of 2.07 TOPS/W (trillions of operations per watt) at 1.0 V supply and a maximum frequency of 52.15 MHz.

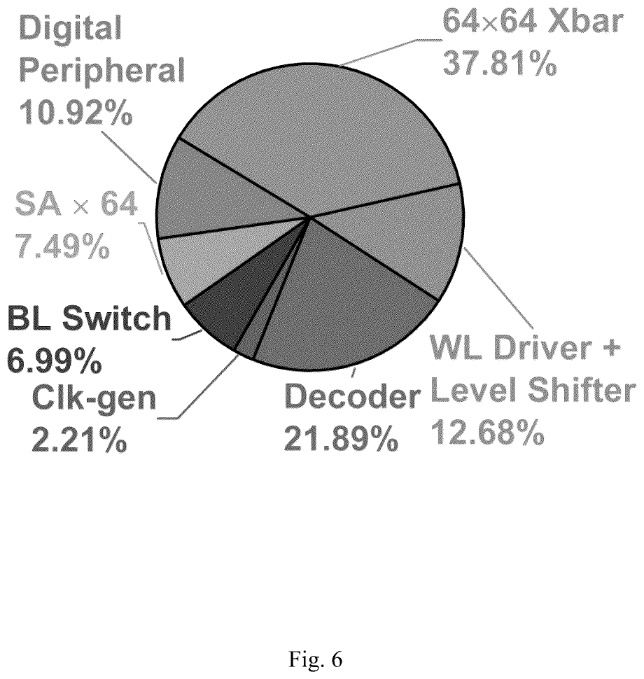

FIG. 6 shows a graphical breakdown of the surface area occupied by each of the components of the disclosed design. Table 1 below includes a summary of the disclosed design.

TABLE 1

| | Disclosed Design |
|---|---|
| Technology Node | 65 nm |
| RRAM Type | 1T1R H fO₂ |
| RRAM Array Size | 64 × 64 |
| Core Design Area (mm²) | 0.1436 |
| Operating Voltage (V) | 0.9~1.2 |
| Operating Frequency (MHz) | 23.7~84.5 |
| Energy Efficiency (TOPS/W) | 2.07 (at 1.0 V) |

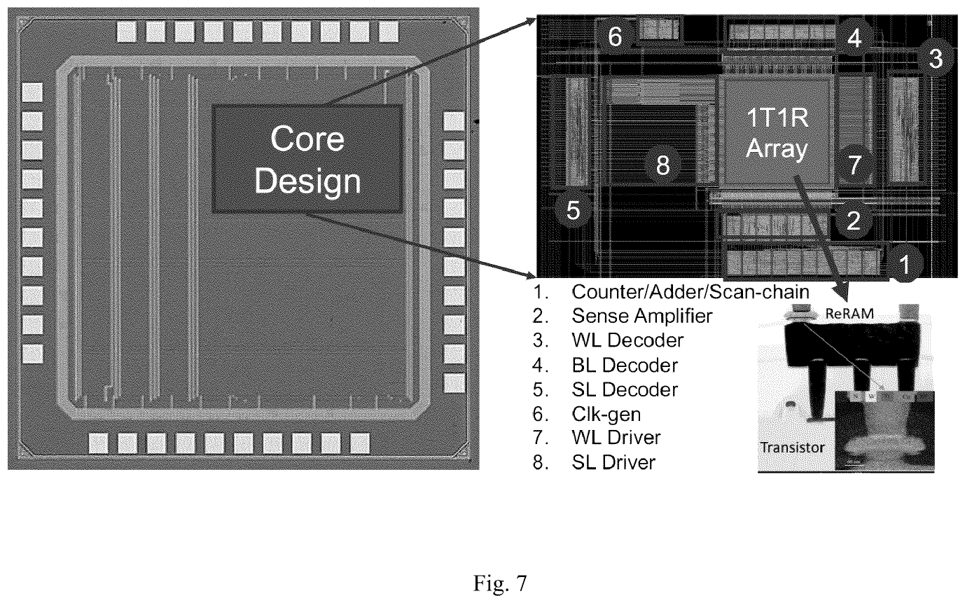

FIG. 7 shows a diagram of the on-die layout of the various components of one exemplary implementation of the disclosed design, and FIG. 8 is a photograph of the experimental setup used to make the measurements disclosed herein.

Table 2 below shows the comparison of the disclosed chip design with four different types of genome alignment platforms: CPU/GPU as general purpose processors, FPGA implementation, and ASIC design. The CPU, GPU, and ASIC data were reported in Y.-C. Wu et al. IEEE TBioCAS, 2017, and the FPGA data was reported in J. Arram et al. IEEE/ACM TCBB, 2017. While CPUs and GPUs run at higher frequencies and have more on-chip memory, the ‘memory-wall’ limits their absolute throughput and energy efficiency. An FPGA-based implementation achieves higher performance due to its large-scale (8 FPGAs in the disclosed implementation) and dedicated dataflow graph. The only related prior CMOS ASIC design shows much improved performance, particularly in terms of throughput-to-area ratio, compared with CPUs/GPUs and FPGAs. Benefiting from the unique CIM architecture, the proposed CIM macro achieves the best performance in all aspects, particularly in energy efficiency and throughput-to-area ratio. Leveraging the high parallelism and reduced data movement of the disclosed CIM architecture, the design of the present disclosure achieves 41.6 higher throughput and 5.73 energy efficiency improvement when measured against the state-of-the-art CMOS ASIC design.

TABLE 2

| | CPU | GPU | | | |
|---|---|---|---|---|---|
| | AMD | NVIDIA | | ASIC | Present |
| Metrics | Opteron 6128 | Tesla M2075 | FPGA | CMOS | Disclosure |
| Technology | 45 nm | 40 nm | 28 nm | 40 nm | 65 nm |
| Die Size | 14.3k | 1.6k | 14.8 | 7.84 | 0.1436 |
| (mm²) | | | | | |
| Power (W) | 80 | <200 | 247 | 0.135 | 0.01 |
| Frequency | 2000 | 1150 | 200 | 200 | 84.5 |
| (MHz) | | | | | |
| On-Chip | 17,120 | 1,664 | N/A | 384 | 0.5 (1-bit)/1 |
| Memory (KB) | | | | | (2-bit) |
| Throughput | 6.9 × 10⁴ | 8.3 × 10⁵ | 1.5 × 10⁸ | 5.1 × 10⁶ | 2.12 × 10⁸ |
| (suffixes/s) | | | | | |
| Energy | 870 | 4200 | 6.2 × 10⁵ | 3.7 × 10⁸ | 2.12 × 10⁹ |
| Efficiency | | | | | |
| (suffixes/J) | | | | | |
| Throughput- | 200 | 1600 | 420 | 6.4 × 10⁵ | 1.47 × 10⁹ |
| to-Area | | | | | |
| suffixes/s/mm² | | | | | |

The present disclosure presents the first CMOS+RRAM CIM chip for accelerating genome sequencing alignment, showing orders of magnitude improvement in energy efficiency and throughput over CPUs/GPUs and prior non-CIM CMOS ASIC design.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

### REFERENCES

The following publications are incorporated herein by reference in their entirety:

H. Li et al. Fast and accurate short read alignment with burrows-wheeler transform. Bioinformatics, 25:1754-1760, 2009.

M. Alser et al. Accelerating genome analysis: A primer on an ongoing journey. IEEE Micro, 40(05): 65-75, September 2020.

S. Angizi et al. AlignS: A processing-in-memory accelerator for DNA short read alignment leveraging SOT-MRAM. In DAC, pp. 1-6, 2019.

F. Zhang et al. Aligner-d: Leveraging in-dram computing to accelerate dna short read alignment. IEEE JETCAS, 13(1):332-343, 2023.

B. Li et al. Rram-based analog approximate computing. IEEE TCAD, 34(12):1905-1917, 2015.

I. Yeo et al. Resistive memories stack up. Nature Electronics, 5(7):414-415, 2022.

A. Sridharan et al. A 1.23-GHz 16-kb programmable and generic processing-in-SRAM accelerator in 65 nm. In ESSCIRC, 2022.

M. Burrows et al. A block-sorting lossless data compression algorithm. Digital Equipment Corporation technical reports, 124, 1994.

Y.-C. Wu et al. A 135-mW fully integrated data processor for next-generation sequencing. IEEE TBioCAS, 11(6):1216-1225, 2017.

J. Hazra et al. Optimization of switching metrics for CMOS integrated HfO2 based RRAM devices on 300 mm wafer platform. In IMW, 2021.

J. Arram et al. Leveraging FPGAs for accelerating short read alignment. IEEE/ACM TCBB, 14(3):668-677, 2017.

What is claimed is:

1. A system for calculating DNA short-read alignment, comprising:
a memory comprising a plurality of memory cells arranged in columns and rows, each column having a bitline, the memory comprising first and second regions;
a plurality of transmission gates, each being connected to a row in the memory;
a plurality of sense amplifiers, each having first and second outputs connected to each bitline in the memory;
a plurality of logic gates, each connected to the first and second outputs of each sense amplifier;
a digital peripheral circuit connected to the outputs of the logic gates; and
a processor communicatively connected to the memory, configured to perform steps comprising:
performing a Burrows-Wheeler transform on a nucleotide sequence represented as a string;
storing the transformed nucleotide sequence in the first region of the memory;
storing a short-read nucleotide sequence in the second region of the memory;
activating first and second rows of the memory via first and second transmission gates of the plurality of transmission gates, wherein the first row is in the first region of the memory and the second row is in the second region of the memory; and
calculating DNA short-read alignment using the digital peripheral circuit.

2. The system of claim 1, wherein the memory is a resistive random-access memory (RRAM).

3. The system of claim 1, wherein the digital peripheral circuit comprises a plurality of latches, each connected to an output of a logic gate.

4. The system of claim 1, wherein the digital peripheral circuit comprises an adder connected to the latches.

5. The system of claim 1, wherein the logic gates are AND gates.

6. The system of claim 1, wherein each memory cell comprises a memristor, and wherein the memristors form a voltage divider with the corresponding bitline when the transmission gates activate the first and second rows.

7. The system of claim 1, wherein the memory, the transmission gate, the sense amplifiers, the logic gates, and the digital peripheral circuit are all positioned on the same integrated circuit.

8. The system of claim 7, wherein the processor is positioned on the same integrated circuit.

9. The system of claim 1, wherein the first transmission gate is configured to connect the first memory row to a source voltage line, and the second transmission gate is configured to connect the second memory row to a ground.

10. The system of claim 1, wherein the memory further comprises a third region, and wherein the processor is configured to store a marker table having an adder for each nucleotide.

11. A method of calculating DNA short-read alignment, comprising:
providing a memory and a processor;
performing a Burrows-Wheeler transform on a nucleotide sequence represented as a string;
storing the transformed nucleotide sequence in the memory;
storing a short-read nucleotide sequence in the memory;
activating first and second rows of the memory wherein the first row holds a portion of the transformed nucleotide sequence and the second row holds a portion of the short-read nucleotide sequence; and
calculating DNA short-read alignment using voltage levels measured at the bitlines in the memory.

12. The method of claim 11, wherein the memory is a resistive random-access memory (RRAM).

13. The method of claim 11, further comprising the step of calculating whether first and second nucleotides stored in the first and second rows are the same by calculating a logical XNOR of the voltage values measured via sense amplifiers at the bitlines.

14. The method of claim 13, wherein the logical XNOR is calculated by comparing a voltage at a junction of a voltage divider formed by resistors in the first and second rows of the memory to first and second different voltage references using first and second sense amplifiers.

15. The method of claim 14, wherein the first voltage reference is at least 10% higher than a midpoint of a supply voltage of the first and second sense amplifiers, and the second voltage reference is at least 10% lower than a midpoint of the supply voltage.

16. The method of claim 13, further comprising the step of storing the result of each logical XNOR in a latch.

17. The method of claim 16, further comprising adding the values stored in the latches using an adder.

18. The method of claim 11, wherein each memory cell in the memory comprises a memristor, and the method comprises the step of creating a voltage divider with the memristors in the first and second row of each column in the memory.
